(19) United States
(12) Plant Patent Application Publication
(10) Pub. No.: US 2008/0134401 P1
(43) Pub. Date: Jun. 5, 2008

(54) Astilbe plant named 'Key Largo'

(76) Inventor: Hendricus M.J. Holtmaat, Zuidwolde (NL)

Correspondence Address: BIOLOGICAL PATENT SERVICES, LLC, 8424 40th AVE N., NEW HOPE, MN, 55427, US

(21) Appl. No.: 11/633,755

(22) Filed: Dec. 5, 2006

(51) Int. Cl. A01H 5/00

(52) U.S. Cl. PLT/407

(57) ABSTRACT

A new cultivar of Astilbe named ‘Key Largo’, characterized by bright green foliage, abundant bright pink flower flowers, a repeat blooming habit with flowers present up to 8 weeks from late July until late September in the Northwest region of the U.S., a vigorous growth habit, suitable use as both a garden and container plant and hardiness in U.S.D.A. Zones 3 to 9.

### BOTANICAL CLASSIFICATION

Astilbe hybrid

### VARIETAL DENOMINATION

‘Key Largo’

### BACKGROUND OF THE INVENTION

The present invention relates to a new and distinct cultivar of Astilbe hybrid in the Simplicifolia Group of hybrids and will be referred to hereafter by its cultivar name, ‘Key Largo’. ‘Key Largo’ represents a new Astilbe, an herbaceous perennial grown for landscape use.

‘Key Largo’ was selected by the inventor in summer of 2000 from a controlled breeding program in Zuidwolde, The Netherlands. ‘Key Largo’ was selected from a cross made between the seed parent Astilbe ‘Sprite’ (not patented), an Astilbe hybrid in the Simplicifolia Group of hybrids and the pollen parent Astilbe ‘Purple Kerze’ (not patented), an Astilbe×arendsii hybrid.

Asexual reproduction of the new cultivar was first accomplished by in vitro propagation in Enkhuizen, The Netherlands under the direction of the inventor in 2000. It has been determined by propagation both by division and tissue culture that the characteristics of the new cultivar are stable and are reproduced true to type in successive generations.

### SUMMARY OF THE INVENTION

The following traits have been repeatedly observed and represent the characteristics of the new cultivar after observing plants grown outdoors in a field for two years in Mossyrock, Wash. These attributes in combination distinguish ‘Key Largo’ as a unique cultivar of Astilbe.
1. ‘Key Largo’ bright green, healthy appearing foliage.
2. ‘Key Largo’ blooms heavily with 5 to 7 flower stems per 2 to 3 eye division.
3. ‘Key Largo’ exhibits flowers that are bright pink in color.
4. ‘Key Largo’ has a repeat blooming habit due to production of new flowering stems, blooming for up to 8 weeks from late July to late September in the upper Northwest section of the U.S.
5. ‘Key Largo’ obtains a height of 40.6 to 45.7 cm (16 to 18 inches) and a spread of 38 cm (15 inches).
6. ‘Key Largo’ is a vigorous grower but suitable for gardens and container growing.
7. ‘Key Largo’ is hardy in U.S.D.A. Zones 3 to 9.

‘Key West’ differs from the seed parent, ‘Sprite’, in that it is a repeat bloomer producing a greater number of flower stems and by exhibiting a more vigorous growth habit.

‘Key Largo’ differs from the pollen parent, ‘Purple Kerze’, in that ‘Key Largo’ has a greater repeat bloom habit, is more compact, begins flowering earlier in the season, and exhibits pink flowers whereas ‘Purple Kerze’ exhibits lavender flowers.

In comparison to ‘Key West’ (plant patent pending), ‘Key Largo’ is slightly taller, has leaves that are green rather than red and bronze-green, and the flowers of ‘Key Largo’ are pink through out development whereas the flowers of ‘Key West’ emerge red and fade to pink.

### BRIEF DESCRIPTION OF THE PHOTOGRAPHS

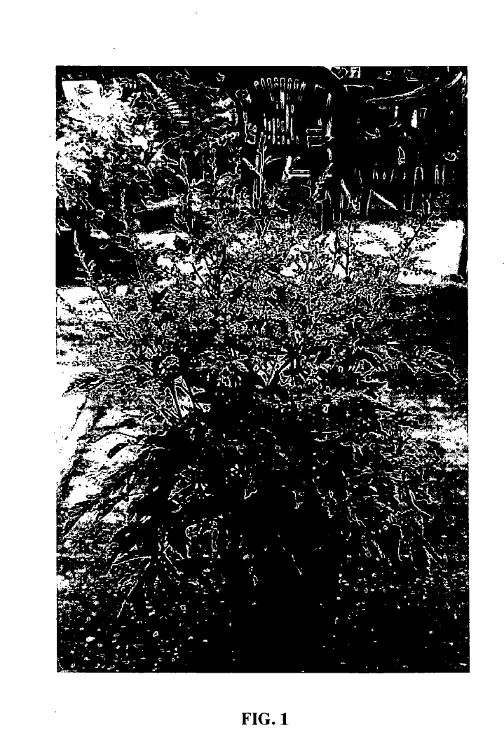

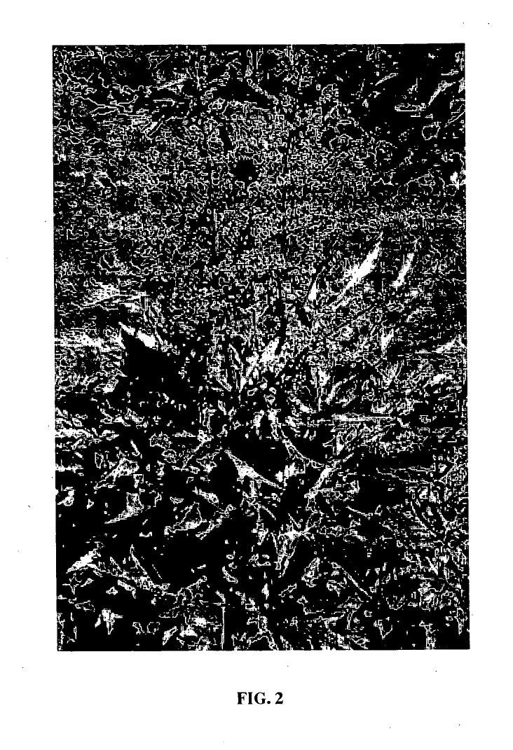

The accompanying colored photographs illustrate the overall appearance and distinct characteristics of the new Astilbe. The plants in the photographs were grown for one year from a 2 to 3 eye division in Mossyrock, Wash. The photograph in FIG. 1 provides an overall view of a plant of ‘Key Largo’ as grown outdoors in a one-gallon container. The photograph in FIG. 2 provides a closer view of the flowers of ‘Key Largo’ on a plant that was field grown. The colors in the photographs are as close as possible with the photographic and printing technology utilized. The color values cited in the detailed botanical description accurately describe the colors of the new Astilbe.

### DETAILED BOTANICAL DESCRIPTION

The following is a detailed description of the new cultivar as grown outdoors in one gallon containers in Mossyrock, Wash. The phenotype of the new cultivar may vary with variations in environmental, climatic, and cultural conditions, as it has not been tested under all possible environmental conditions. The color determination is in accordance with the 2001 R.H.S Colour Chart of The Royal Horticultural Society, London, England, except where general color terms of ordinary dictionary significance are used.
Botanical classification: ‘Key Largo’ is a cultivar of hybrid Astilbe in the Simplicifolia Group of hybrids.
General description:
   Blooming period.—Repeat bloomer, blooming up to 8 weeks from late July into late September in the Northwestern region of the U.S.
   Plant type.—Herbaceous perennial.
   Plant habit.—Clump-forming, upright flowering stems with cascading foliage, relatively compact.
   Height and spread.—Reaches 40.6 to 45.7 cm (16 to 18 inches) in height and about 38 cm (15 inches) in width.
   Hardiness.—U.S.D.A. Zone 3 to 9.
   Culture.—Prefers humus-rich, moist but well-drained soils in partial shade or full sun in areas where summers are cool with sufficient moisture.
   Diseases and pests.—Astilbe is typically disease free, foliage maintains a healthy appearance.
   Root description.—Fibrous, emerging from rhizome.
Growth and propagation:
   Propagation.—Rhizome Division and tissue culture.
   Growth rate.—Moderate growth rate.
   Cropping time to bloom.—A bare root 2 to 3 eye division will finish in a one-gallon container in about 90 days when grown outdoors with an average temperature of 12° C. under ambient light.
Stem description:
   Stem shape.—Slightly ovoid, swollen at nodes and base of petiole.
   Stem color.—Young stems; a color between 144A and 144B, mature stems; 144A suffused with 178A.
   Stem size.—About 24 cm in length and ranges from 2.5 cm to 5 cm in diameter.
   Stem surface.—Sparsely pillose with hairs primarily 178A in color.
   Stem aspect.—Strong, held upright to 45° angle.
   Internode length.—Varies from 3.5 cm to 10 cm between lateral branches.
   Branching habit.—Branched with 2 to 3 lateral branches per main stem from crown.
Foliage description:
   Leaf division.—Bi-ternate.
   Leaf attachment.—Petiolate.
   Leaf arrangement.—Alternate.
   Leaf size.—Average of 11 cm in length and 13 cm in width.
   Leaflet shape.—Terminals; ovate to broadly lanceolate, laterals; primarily broadly lanceolate.
   Leaflet base.—Rounded to cordate.
   Leaflet apex.—Attenuate.
   Leaflet venation.—Pinnate, color is the same as the leaf color on upper surface, raised and 145A in color on lower surface.
   Leaflet margins.—Biserrate.
   Leaflet size.—Lateral leaflets; up to 4.5 cm in length and 3.5 cm in width, terminal leaflets; up to 6 cm in length and 5 cm in width.
   Leaflet surface.—Upper surface; semi glossy, lower surface; sparsely pubescent and rough.
   Leaflet color.—New foliage upper surface; 137B, new foliage lower surface, 137D, mature foliage upper surface; 139A, mature foliage lower surface; 137C.
   Petioles.—144B in color, about 2 to 3 mm in diameter and ranges from about 8 to 10 cm in length, surface is pubescent.
   Petiolules.—144B in color, about 1 mm in width and 2 to 4 cm in length for the primary petiolules and 4 mm to 1 cm in length for the secondary petiolules, surface is pubescent.
Flower description:
   Inflorescence type.—Numerous rotate single flowers arranged on broadly pyramidal branched panicles.
   Inflorescence size.—Reaches up to 20 cm in height and about 18 cm in width in full bloom.
   Flower fragrance.—None detected.
   Flower quantity.—Average of 3 flowering stems per one gallon sized plant, approximately 1,000 flowers per flowering stem.
   Flower lastingness.—Average of 10 days per flower, individual panicles blooms for about 3 weeks, flowers persistent.
   Flower buds.—Globose in shape, average of 1.5 mm in diameter and 2.5 mm in length, 144A to 144B in color suffused with N81A towards apex.
   Flower aspect.—Held at about a 45° angle from Rachis.
   Flower shape.—Bell shaped.
   Flower size.—About 4 mm in depth and 2 mm in diameter.
   Petals.—About 5, linear and twisted in shape, margin is entire, apex is acute, upper and lower surface is glabrous, color of upper and lower surface is 73A to 73B, about 3 mm in length and 0.5 mm in width.
   Calyx.—Campanulate, sepals fused at base.
   Sepals.—5, elliptic in shape, margin is entire, apex is acute, base is fused, surface is smooth in appearance, color of upper and lower surface is 144A to 144B suffused with N81A towards apex, about 1 mm in width and 2 mm in length.
   Bracts.—2, lanceolate in shape, 144B in color, unequal with one about 1 mm in width and 2 mm in length and the other about 0.75 mm in width and 1 mm in length, attenuate apex, base fused to rachis, 144B in color.
   Peduncles.—About 6 to 10 cm in length and an average of 2.5 mm in width, held upright at an angle of about 45° relative to stem, 144A suffused with 178A in color.
   Pedicels.—About 0.5 mm in length and 0.5 mm in width, 144A suffused with 178A in color.
   Rachis.—About 20 cm in length with secondary branches about 7 mm in length, up to 3 mm in width with secondary branches about 1.5 mm in width, 144A suffused with 178A in color.
Reproductive organs:
   Gynoecium.—2 Pistils, club-shaped, about 2 mm in length and one mm in width, stigma 178A in color and minute, style is about 1 mm in length and 1.5 mm in width at base, translucent and 157D in color, ovary is about 1 mm in width and length, superior and 144D in color.
   Androcoecium.—10 stamens, anthers are oblong in shape, basifixed, about 0.2 mm in length and width and 197C in color, filaments are about 1.5 mm in length and very fine, pollen is not visible.
   Seed.—Seed development has not been observed.

I claim:

1. A new and distinct cultivar of Astilbe plant named ‘Key Largo’ as herein illustrated and described.
